(19) United States
(12) Patent Application Publication
(10) Pub. No.: US 2014/0031874 A1
(43) Pub. Date: Jan. 30, 2014

(54) MINIMALLY INVASIVE DEVICES, SYSTEMS AND METHODS FOR TREATING THE SPINE

(71) Applicant: Spinal USA, Inc., Parisppany, NJ (US)

(72) Inventors: Donald Kucharzyk, Crete, IL (US); Stephen Termyna, Boonton, NJ (US); Thomas W. Winegar, Hawthorne, NJ (US); Luis A. Arellano, Fair Lawn, NJ (US); John Wilson, Brandon, MS (US)

(73) Assignee: Spinal USA, Inc., Parisppany, NJ (US)

(21) Appl. No.: 13/952,324

(22) Filed: Jul. 26, 2013

Related U.S. Application Data
(60) Provisional application No. 61/676,856, filed on Jul. 27, 2012.

(51) Int. Cl. A61B 17/70

(52) U.S. Cl. CPC A61B 17/7076
USPC 606/279; 600/210; 606/104

(57) ABSTRACT

Devices and methods are provided for surgical retraction with a minimally invasive, maximum access surgical system. The surgical system can include anchor extensions that can be attached to bone screws. The bone screws can be inserted into a pedicle of a vertebral body. A retractor can be attached to anchor extensions connected to adjacent vertebrae on an operational side, and the retractor can be attached to anchor extensions connected to adjacent vertebrae on a contralateral side. The retractor can be used to distract the vertebral disc space between the adjacent vertebrae.

### CROSS-REFERENCE TO RELATED APPLICATIONS

Any and all applications for which a foreign or domestic priority claim is identified in the Application Data Sheet as filed with the present application, are hereby incorporated by reference under 37 CFR 1.57. This application is related to and claims priority to U.S. Provisional Application No. 61/676,856, filed Jul. 27, 2012, the entire application of which is hereby incorporated by reference and made a part of this specification.

### BACKGROUND OF THE DISCLOSURE

1. Field of the Disclosure

The present application relates to devices, systems and methods for treating the spine. In certain embodiments, the present application relates to devices, systems and methods for providing spinal stabilization, such as a spinal fusion. In particular, certain embodiments relate to minimally invasive devices and methods for delivering fixation devices and implants into the spine.

2. Description of the Related Art

Spinal bone and disc degeneration can occur due to trauma, disease or aging. Such degeneration can cause abnormal positioning and motion of the vertebrae, which can subject nerves that pass between vertebral bodies to pressure, thereby causing pain and possible nerve damage to a patient. In order to alleviate the pain caused by bone degeneration, it is often helpful to maintain the natural spacing between vertebrae to reduce the pressure applied to nerves that pass between vertebral bodies.

To maintain the natural spacing between vertebrae, spinal stabilization devices are often provided to promote spinal stability. These spinal stabilization devices can include fixation devices, such as spinal screws, which are implanted into vertebral bone. The fixation devices work in conjunction with other implanted members, such as rod members, to form stabilization systems.

Spinal stabilization devices are often used in conjunction with spinal fusion techniques, which can increase stability of the spacing between vertebrae by fusing adjacent vertebrae together. Two of the most common spinal fusion techniques are the transforaminal lumbar interbody fusion (TLIF) and the posterior lumbar interbody fusion (PLIF).

Conventional stabilization systems and techniques often require open surgeries and other invasive procedures in order to deliver the implants into the body. These invasive procedures often cause a great deal of pain and trauma to the patient, and require a substantial recovery time. Minimally invasive (MIS) and maximal access (MAS) systems and methods exist, but they are relatively new and with room for improvement.

### SUMMARY OF THE DISCLOSURE

Various embodiments described herein relate to a minimally invasive retractor access system that can be used as part of a minimally disruptive muscle sparing approach to the spine. Embodiments described herein can be used for single and multi-level spinal fusions. Embodiments described herein can also be used for bilateral distraction of a spinal disc space during a spinal procedure, such as by distracting from devices attached to bone screws on both sides of a spine. Among other benefits, this can help when distracting tight disc spaces.

In some embodiments, a minimally invasive surgical system for treating the spine can include at least one blade assembly. The blade assembly can have a blade with a central section having a central bore and wings extending outward from the central section on opposite sides thereof. The wings also extend to a location below a bottom of the central section, forming a gap bounded by the wings on two sides and the bottom of the central section on a third side. The blade assembly can also have a post extending from an upper surface of the blade and a shaft positioned at least partially within the central bore of the blade, the shaft having an externally threaded lower section and a central shaft bore that runs through the length of the shaft. The shaft can be rotatable within the central bore of the blade.

In some embodiments, the blade assembly can include an attachment section extending from the blade. In some embodiments, the shaft can have a plurality of vertical grooves extending around at least a portion of the shaft. In some embodiments, the blade assembly can further include a locking pin positioned within the attachment section, the locking pin moveable between a locked position in which a point on one end of the locking pin engages at least one of the vertical grooves of the blade shaft, substantially preventing rotation of the shaft, and an unlocked position in which the point is disengaged from the vertical grooves. In some embodiments, the locking pin can also be biased toward the locked position. In some embodiments, the vertical grooves are angled relative to a diameter of the shaft bore such that the locking pin in the locked position only prevents rotation of the shaft in one direction.

In some embodiments, the externally threaded lower section extends at least partially into the gap. In some embodiments, the surgical system can further include a pedicle screw having a screw shank and a housing attached to an upper end of the screw shank. The housing can have internal threading configured to receive the external threads of the lower section of the blade shaft.

In some embodiments, the surgical system can further include a retractor having a cross bar with a plurality of arms extending from the cross bar. In some embodiments, at least one of the arms can move relative to the cross bar to thereby change a distance between the arms. In some embodiments, each arm at its end farthest from the cross bar can have a collar configured to attach to the post of the blade assembly. In some embodiments, an attachment assembly can be connected to one of the arms, the attachment assembly configured to attach to a minimally invasive tower access device.

In some embodiments, the retractor can have a first set of arms extending from the cross bar on a first side of the cross bar, and a second set of arms extending from the cross bar on a second side of the cross bar. Each arm of the first set can have at least one articulating section. The arms of the first set can be moved closer together or further apart and the arms of the second set can be moved closer together or further apart. In some embodiments, each arm of the second set can be configured to attach to a minimally invasive tower access device. In some embodiments, when the arms of the first set are attached to blade assemblies that are attached to pedicle screws positioned within pedicles on the first side of a pair of adjacent vertebrae, and when the arms of the second set are attached to minimally invasive tower access devices that are attached to pedicle screws positioned within pedicles on the second side of the pair of adjacent vertebrae, the arms of each set can be simultaneously moved further apart to distract the disc space between the pair of adjacent vertebrae.

In some embodiments, a medial blade assembly can be configured to attach to at least one of the aims, the medial blade assembly comprising a medial blade configured to be movable toward the cross bar. In some embodiments, the medial blade assembly is movable in a direction toward at least one of the arms.

In some embodiments, a minimally invasive surgical system for treating the spine includes a plurality of pedicle screws for insertion into the pedicles of adjacent vertebrae, with each of the pedicle screws having an upper portion that is internally threaded. The system can also include a plurality of retractor blades, each of the blades having an externally threaded lower section configured to engage the internally threaded upper portion of a corresponding pedicle screw. The system can further include a plurality of retractor arms configured to move the retractor blades closer together or farther apart. In some embodiments, the system can further include a plurality of minimally invasive tower access devices configured to engage at least some of the plurality of pedicle screws. In some embodiments, the plurality of retractor arms includes at least some retractor arms configured to engage the plurality of minimally invasive tower access devices to move the minimally invasive tower access devices closer together or farther apart. In some embodiments, the system can further include an implant configured to be delivered between the plurality of retractor blades or between the plurality of tower access devices.

In various embodiments, a minimally invasive surgical system for treating the spine can include four pedicle screws, each pedicle screw configured to be positioned in a respective pedicle of a first vertebra and a second vertebra adjacent the first vertebrae. The system can also include four pedicle screw extensions, each extension configured to engage a respective pedicle screw. The system can further include a retractor body having a cross bar, a plurality of aims extending from the cross bar with at least two of the arms movable relative to each other, and four connection assemblies, each connection assembly attached to an arm and configured to attach to a respective pedicle screw extension and pedicle screw. In some embodiments, moving the at least two arms apart from each other moves the connection assemblies attached to pedicle screws positioned in the pedicles of the first vertebra apart from the connection assemblies attached to pedicle screws positioned in the pedicles of the second vertebra.

In some embodiments, the retractor body can include two arms and each arm can be attached to two connection assemblies. In some embodiments, two of the pedicle screw extensions can be blade assemblies and two of the pedicle screw extensions can be tower access devices. In some embodiments, the plurality of arms can extend from the cross bar on the same side of the cross bar.

In various embodiments, a screwdriver and screw extender can be used to help attach screws used in spinal procedures. The screwdriver can have a section with ridges, and the screw extender can have a moveable locking plate with an edge or tooth configured to releasably engage the ridged section of the screwdriver. In some embodiments, the locking plate can be biased into engagement with the screwdriver. In some embodiments, a user can manually release the locking plate from engagement with the screwdriver. In some embodiments, a screw extender can have multiple locking plates or other components configured to releasably engage the screwdriver.

Methods are also described herein. In some embodiments, a minimally invasive surgical method for treating the spine includes delivering a first pedicle screw into the pedicle of a first vertebra and a second pedicle screw into the pedicle of a second vertebra, the first and second pedicle screws having threaded upper portions. The method can also include distracting a disc space between the first and second vertebra by moving retractor blades engaged with the threaded upper portions of the first and second pedicle screws, and delivering an implant between the retractor blades into the disc space. In some embodiments, the method can further include delivering a third pedicle screw into the pedicle of the first vertebra on a contralateral side relative to the first pedicle screw, and delivering a fourth pedicle screw into the pedicle of the second vertebra on a contralateral side relative to the second pedicle screw. In some embodiments, the disc space further can be distracted at least in part by moving screw extensions engaged with the third and fourth pedicle screws. In some embodiments, the screw extensions can be minimally invasive access towers. In some embodiments they can be retractor blades. In some embodiments, the disc space can be distracted by simultaneously moving the retractor blades and screw extensions.

### BRIEF DESCRIPTION OF THE DRAWINGS

FIG. 1A is a perspective view of one embodiment of a retractor system for use in a minimally invasive surgical system.

FIG. 1B is a perspective view of one embodiment of a retractor system for use in a minimally invasive surgical system.

FIG. 1C is a perspective view of one embodiment of a retractor system for use in a minimally invasive surgical system.

FIG. 2 is a perspective view of one embodiment of a pedicle screw with a rod and set screw.

FIG. 3 is a perspective view of one embodiment of a retractor blade of a retractor blade assembly.

FIG. 4 is a cross-sectional side view of the retractor blade of FIG. 3.

FIG. 5 is a perspective view of one embodiment of a shaft of a retractor blade assembly.

FIG. 6 is a cross-sectional side view of the shaft of FIG. 5.

FIG. 7 is a perspective view of one embodiment of a retractor blade assembly.

FIG. 8A is a top cross-sectional view of the retractor blade assembly of FIG. 7.

FIG. 8B is a perspective view of the retractor blade assembly of FIG. 8A, with the retractor blade transparent.

FIG. 9A is a side view of one embodiment of a screw extender.

FIG. 9B is a side view of the screw extender of FIG. 9A, rotated 90 degrees.

FIG. 9C is a cross-sectional view of the screw extender of FIG. 9A.

FIG. 10 is a side view of one embodiment of a screwdriver.

FIG. 11 is a top cross-sectional view of one embodiment of a screw extender.

FIG. 12 is a side view of one embodiment of a screwdriver.

FIG. 13A is a top view of one embodiment of a locking plate.

FIG. 13B is a side cross-sectional view of the locking plate of FIG. 13A.

FIG. 14 is a cross sectional view of a section of a screwdriver and screw extender that can lock a screwdriver on two sides.

FIG. 15 is a perspective view of one embodiment of an extension attachment assembly.

FIG. 16 is a top view of the extension attachment assembly of FIG. 15.

FIG. 17 is a cross-sectional view of the extension attachment assembly of FIG. 15.

FIG. 18 is a perspective view of one embodiment of a medial blade attachment assembly.

FIG. 19A is a side view of the medial blade attachment assembly of FIG. 18.

FIG. 19B is a cross-sectional view of the medial blade attachment assembly of FIG. 18.

FIG. 20 is a perspective view of one embodiment of a medial blade attachment assembly.

FIG. 21 is a perspective view of one embodiment of a medial blade attachment assembly.

FIG. 22 is a perspective view of one embodiment of a medial blade attachment assembly.

FIG. 23 is a perspective view of one embodiment of a medial blade attachment assembly.

FIG. 24 is a perspective view of one embodiment of a minimally invasive tower access device.

FIG. 25 is a perspective view of one embodiment of a retractor body.

FIG. 26 is a perspective view of one embodiment of a retractor body.

### DETAILED DESCRIPTION OF THE PREFERRED EMBODIMENTS

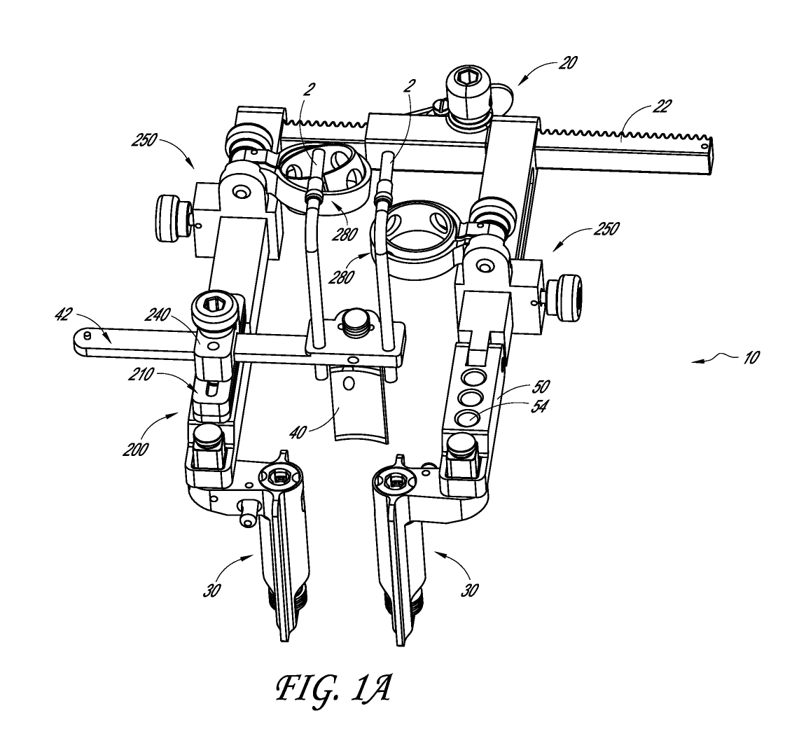

FIG. 1A illustrates an embodiment of a retractor system 10 used as part of a minimally invasive (MIS) and/or maximum access (MAS) spinal surgery. This disclosure may refer to MAS or MIS surgeries with respect to certain embodiments, but it is to be understood that such use does not preclude any embodiment described herein from being considered an MIS and/or an MAS surgery.

In some embodiments, the minimally invasive (MIS) and maximal access (MAS) surgical techniques and apparatuses disclosed herein can include a retractor body 20 with a plurality of arms 50. The device as illustrated has two arms extending from a cross bar 22 of the retractor body. Generally, during a surgical procedure the cross bar 22 can be positioned above a patient's back and the arms 50 can point laterally on an operational side of the patient's spine. As used throughout the present disclosure, the “operational side” shall refer to a side of the patient from which disc space of the spine is accessed (e.g., for delivery of a spinal implant). In some embodiments, the cross bar 22 can be positioned generally directly above the patient's spine or on the operational side of the patient's spine and the arms can point laterally. In some embodiments, the cross bar 22 can be positioned on a contralateral side (i.e., a side opposite the operational side) of the patient's spine and the arms can extend across the spine. In some embodiments, the cross bar 22 can be positioned above or adjacent the spine and the retractor body can have two arms extending laterally on the contralateral side and two arms extending laterally on the operational side, for a total of four arms. In some embodiments, the retractor body can have more than two arms on one or both sides.

The arms 50 can be configured to attach to anchor extensions, such as lateral blade assemblies 30, which can each be configured to attach to and extend from a bone anchor (typically a bone screw, such as a pedicle screw). Bone screws can attach to blade assemblies such that the shaft of the screw extends below the bottom edge of the blade. “Bottom” or “below” as used herein are with reference to the anterior side of the patient when the disclosed devices and components are positioned on or in the patient during surgery. Thus, the bottom edge of a blade assembly 30 is the edge that would face the anterior side of the patient. The lateral blade assemblies 30 can be used to retract tissue during a surgical procedure. Additionally, because the lateral blades are attached to the pedicle screws, and because they can be configured to move with the arms in either direction along a generally caudal-cranial line when positioned in a patient (as described further below), they can also help distract disc space when the pedicle screws are screwed into adjacent vertebrae. Other types of anchor extensions can similarly be used to distract a disc space.

In some embodiments, a medial blade assembly 200 can be attached to the retractor body 20, such as to one or more of the arms 50. The medial blade 40 can be configured to move in a medial-lateral direction when placed within a patient, helping to retract tissue and create a clear view of the vertebrae. In some embodiments, the medial blade assembly can attach to the arms through attachment holes 54. As described in more detail below, the medial blade assembly can include an assembly attachment portion 210, a medial blade positioning portion 240, and a medial blade arm 42 that can attach to a medial blade 40.

In some embodiments, a retractor system can also include one or more anchor extension attachment assemblies 250. The extension attachment assemblies can be used to attach the retractor system to a variety of anchor extensions or surgical devices, such as one or more minimally invasive surgical (MIS) towers, as discussed further below. In some embodiments, a retractor body 20 can be positioned such that lateral blade assemblies 30 are on one side (e.g., an operational side) of a patient's spine and extension attachment assemblies 250 are on another side (e.g., a contralateral side) of the patient's spine.

Where the lateral blade assemblies are attached to pedicle screws on the operational side and the extension attachment assemblies interface with extensions (such as MIS towers) attached to pedicle screws on the contralateral side, separating the arms 50 of the retractor body 20 can create bilateral distraction, distracting the disc space on both sides of the spine. In some embodiments, the arms can be generally parallel to each other, such that the bilateral distraction creates a generally equal amount of distraction on both sides of the spine. In some embodiments, the arms can be at an angle relative to one another, such that bilateral distraction creates an amount of distraction on a first side of the spine that differs from the amount of distraction on a second side of the spine.

Having the retractor system 10 attached to anchor extensions on both sides of the spine creates a number of advantages. For example, the retractor system will be better anchored into the body. In some embodiments, the anchoring can be such that the system does not need to be anchored to the operating table or other external support structure, as is typically done. Further, where two screws are inserted into the operational side (one each on two adjacent vertebrae) and corresponding screws are inserted on the contralateral side, the retractor system can distribute forces between four screws instead of just two. Additionally, by allowing for distraction on both sides of the disc space, the retraction system described herein can be used to perform a TLIF, TPLIF, PLIF or other procedure according to the surgeon's choice without having to vary the setup.

In some embodiments, a surgeon can modify the device from the illustrated embodiment according to preference. For example, in some embodiments a surgeon may attach four extension attachment assemblies 250 to the arms 50 of the device. This could allow, for example, the surgeon to attach the retractor body to MIS towers on an operational side of a patient's spine and to MIS towers on a contralateral side of the patient's spine. Alternatively, in some embodiments a surgeon may elect to have two lateral blade assemblies on a first side of the spine and a lateral blade assembly and an extension attachment assembly on a second side of the spine. In some embodiment, the device may have four lateral blade assemblies. Alternate configurations may include more than two blade assemblies or extension attachment assemblies on one or both sides of the spine.

The extension attachment assembly 250 can include a hoop assembly 280. In some embodiments, the hoop assembly can be configured to connect to an anchor extension at varying angles relative to the arms 50. Additionally, as discussed further below, in some embodiments the extension attachment assemblies 250 can be configured to move along a length of the arm 50 into a desired position.

In some embodiments, it can be desirable to attach devices that allow for improved visibility of a surgical location. In some embodiments, lighting components 2 can attach to a retractor system 10 at any location that helps provide light to the surgical location without unduly obstructing the surgeon's view. For example, in some embodiments lighting components 2 can attach to the blade arm 42, as illustrated. Lighting components can attach to other portions of the retractor system 10 as well.

FIG. 1B illustrates one embodiment of a retractor system 10 without extension attachment assemblies 250 attached to the retractor body 20. In some embodiments, as illustrated, only retractor blade assemblies 30 may be attached to the retractor body.

FIG. 1C illustrates an embodiment of a retractor system 10 without lateral blade assemblies attached. As illustrated, in some embodiments the retractor arms 50 can have a lateral blade connection assembly or collar 56, which can be used to attach the arms to a lateral blade assembly. The collar can have a variety of shapes. In the illustrated embodiment, the collar is configured to attach to a rectangular post of a lateral blade assembly, discussed further below. This can help ensure that the blade assembly is properly oriented when attached to a retractor arm 50. FIG. 1C also illustrates an embodiment of a retractor system in which the extension attachment assemblies 250 are not aligned. Thus, a first attachment assembly can be positioned a first distance from the crossbar 22 and a second attachment assembly can be positioned a second distance from the crossbar 22, such that the first distance is different from the second distance. In some embodiments, the two attachment assemblies can be positioned the same distance from the cross bar 22. In such embodiments, a line perpendicular to both arms 50 can pass through a center of both hoop assemblies 280. In some embodiments, as further illustrated in FIG. 1C, one or more retractor arms 50 do not have attachment holes 54.

### Pedicle Screws

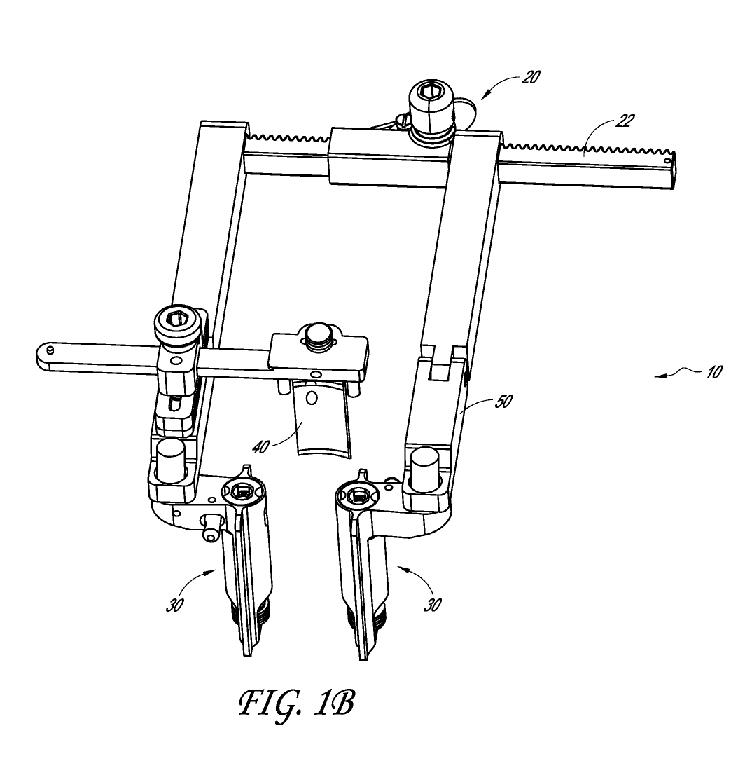

FIG. 2 illustrates a pedicle screw 60 that can be used with a MAS retractor system, as disclosed herein. The pedicle screw can include a cannulated shaft 62 (such that the screw can follow a guide wire) and a housing 70 (also referred to as a head or tulip). The housing can have interior threading 76 and openings 72 extending from the top of the housing on opposite sides. The openings can be sized to receive a rod 64, which can be used to connect multiple pedicle screws and create a spine stabilization system, and which can be locked into place by a set screw 66. Further details of pedicle screws are provided in U.S. Patent Application Publication No. 2010/0241175, published on Sep. 23, 2010, which is hereby incorporated by reference in its entirety and is to be considered part of this specification.

### Retractor Blade Assemblies

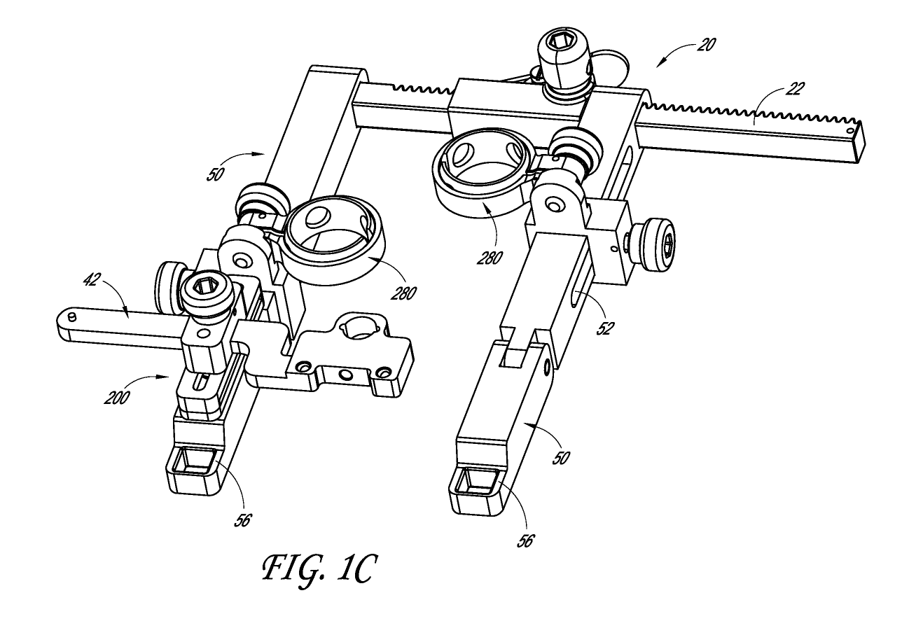

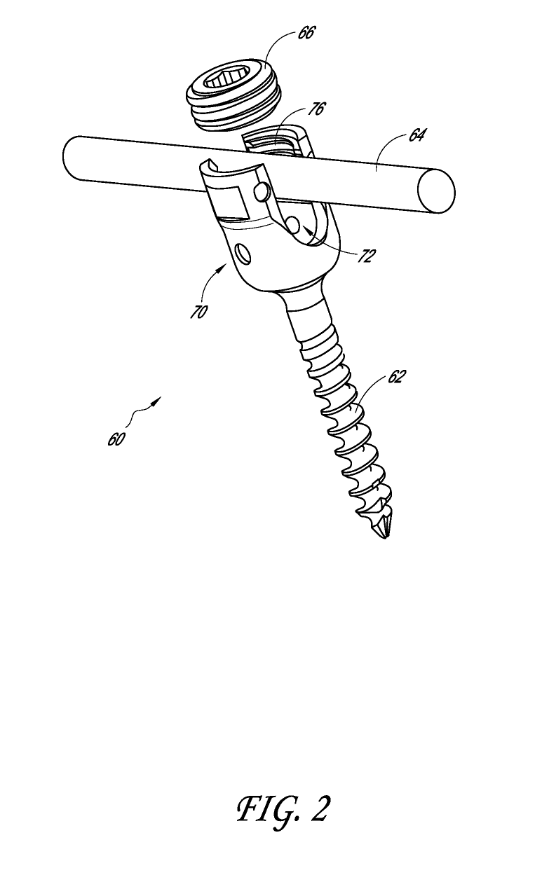

FIGS. 3 and 4 illustrate one embodiment of a retractor blade 80 that can be used with a MAS retractor system as disclosed herein. FIG. 3 is a perspective view of the blade, and FIG. 4 is a cross-sectional view. The retractor blade can comprise an attachment section 90 and a blade section 82, the blade section extending down from the attachment section. The blade section can have a cylindrical central bore 86 within a generally cylindrical central section 84, and the central bore can run from the top of the retractor blade through the bottom of the central section. In some embodiments, the top of the bore can have a larger diameter than lower sections of the bore, creating a ledge 87. In some embodiments, the ledge 87 can be a ramped surface. In some embodiments, the generally cylindrical central section 84 can be square, oval, or of other shapes.

The blade section 82 can have wings or blade extensions 88 that extend outward from opposite sides of the central section 84. The blade extensions 88 can extend below a bottom most point of the central section 84, creating a gap 81 as illustrated. In some embodiments, the blade extensions can extend straight out from the central section 84 in a common, shared plane. In some embodiments, the blade extensions can be curved and/or extend out at an angle from the central section. The blade extensions are preferably symmetric about a line perpendicular to the longitudinal axis of the central bore 86 of the blade 80. For reference purposes, the plane that is perpendicular to the blade extension line of symmetry, and on which the longitudinal axis of the central bore lies, can be referred to as the “blade plane.” In some embodiments, there are no blade extensions extending outward from the central section 84 and the central sections of adjacent retractor blades can provide access to a surgical site, with or without a medial blade assembly 42.

The attachment section 90 can extend from the blade section 82 and can be formed integrally with the blade section. Preferably, the attachment section extends from the blade section in a direction perpendicular to the blade plane. In some embodiments, the attachment section can have a lever bore 92 that extends at least partially into the attachment section from the same side as one of the blade extensions 88. The attachment section can also have a pin bore 96, which can extend into the attachment section from the central bore 86. In some embodiments, a longitudinal axis of the pin bore can be orthogonal to a longitudinal axis of the central bore. In some embodiments, a longitudinal axis of the lever bore 92 can be orthogonal to both the longitudinal axis of the pin bore 96 and the longitudinal axis of the central bore 86. The attachment section can also include a post attachment hole 94, a post pin hole 98, and a lever pin hole 93, as illustrated.

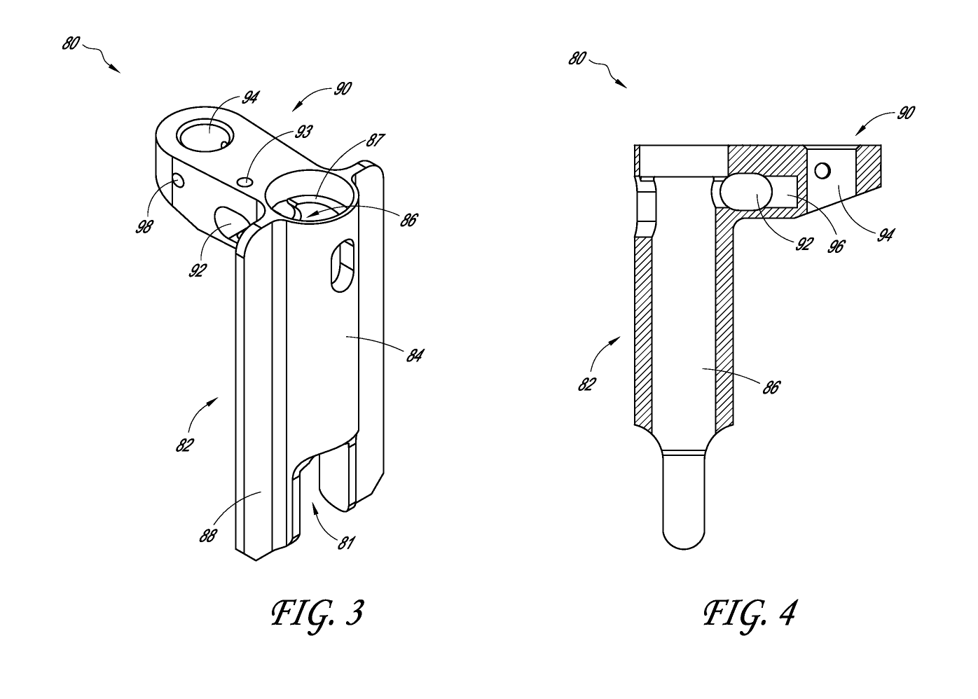

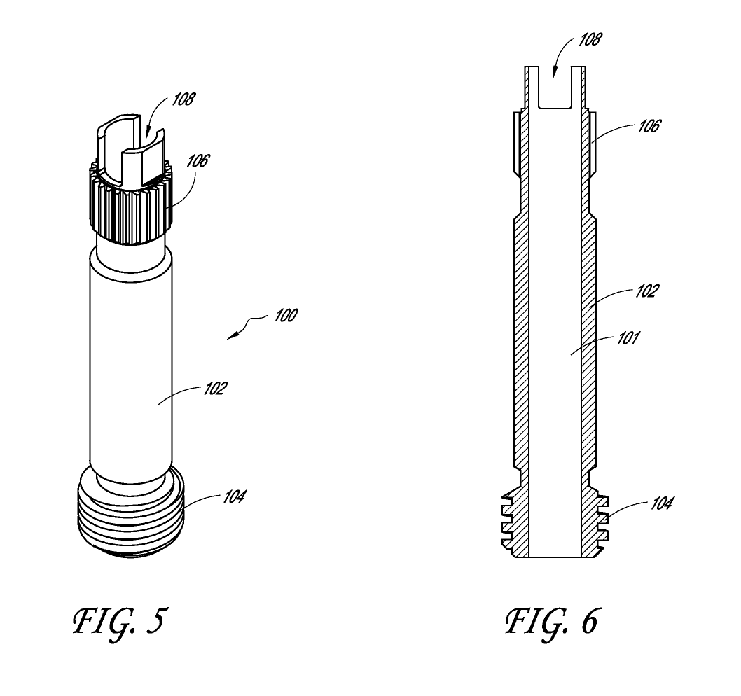

FIGS. 5 and 6 illustrate a retractor blade shaft 100, which can be inserted into the central bore 86 of the retractor blade 80. FIG. 5 is a perspective view of the shaft 100 and FIG. 6 is a cross sectional view. The blade shaft 100 can include a middle shaft section 102, a threaded portion 104 along a bottom section of the retractor blade shaft, and a central shaft bore 101 that extends through the entire length of the shaft. The threaded portion 104 is preferably sized to be able to screw into the tulip of a pedicle screw, mating with the interior threads of the tulip. The retractor blade shaft 100 can also include a plurality of vertical locking grooves 106 extending around the shaft at an upper end thereof. Above the locking grooves 106, the retractor blade shaft can also have a plurality of notches 108, which can be spaced on opposite sides from each other. In some embodiments, the section of the shaft with locking grooves 106 can have substantially the same diameter as the middle shaft section 102. In some embodiments, the middle shaft section 102 and the section with locking grooves 106 can each be sized and configured to fit within the central bore 86 of the retractor blade 80.

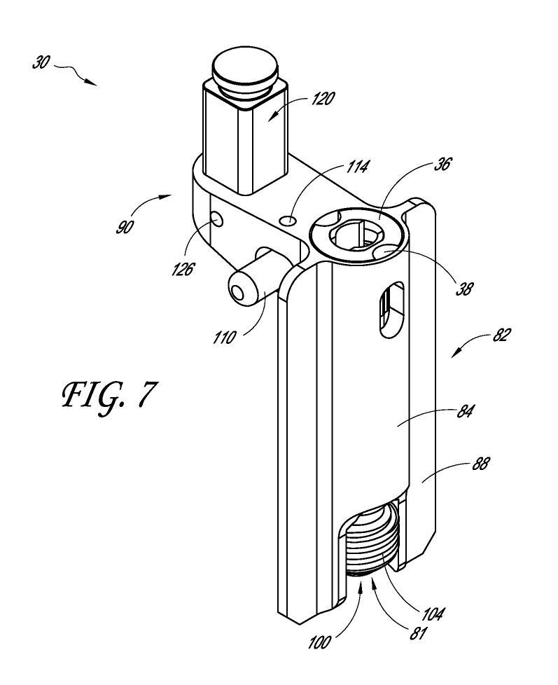

FIG. 7 illustrates a perspective view of a lateral blade assembly 30 in which the retractor blade shaft 100 has been positioned within the central bore of the retractor blade 80. Although not visible in FIG. 7, when the blade shaft is within the retractor blade at least a portion of the locking grooves 106 can be level with at least a portion of the attachment section 90. In some embodiments, the threaded portion 104 of the blade shaft can be wider than the central bore 86, such that to be assembled the retractor blade shaft must be inserted from the bottom of the central bore. The threaded portion 104, however, is preferably sized such that it can fit within the gap 81 of the retractor blade. In some embodiments, the retractor blade shaft 100 has a length such that the threaded portion 104 does not extend all the way to the bottom of the blade extensions 88 when the retractor blade shaft is positioned within the retractor blade 80.

Preferably, the retractor blade 80, the retractor blade shaft 100, and a pedicle screw 60 are all sized and configured such that the threaded bottom 104 of the blade shaft can be screwed into the tulip 70 of the pedicle screw. The blade extensions 88 can be sized such that as the blade assembly 30 is screwed into the tulip the extensions slide into the openings 72 along the sides of the tulip (visible in FIG. 2). This can prevent the tulip from rotating relative to the retractor blade 80.

Because the blade assembly 30 can attach directly to the tulip 70 of the pedicle screw 60, the screw does not have to be installed in a patient in a modular fashion. Consequently, in some embodiments the screw can be installed in a patient as a preassembled or non-modular unit, saving the extra steps of attaching the tulips after performing or during the desired procedure. Further, in some embodiments the retractor blade shaft 100 can rotate within the central bore 86, allowing the blade assembly 30 to be screwed into the tulip 70 of a pedicle screw 60 without rotating the retractor blade 80 itself. Thus, in some embodiments the blade assembly 30 and pedicle screw 60 can be screwed together after the pedicle screw is placed within a vertebra. Further, because the blades can rotate, a single blade can be used regardless of the side of the disc space to which the blade is attached; if the blade needs to be rotated for proper positioning it can easily do so. This also can make it easier to perform surgical procedures on multiple spinal levels, as discussed further below.

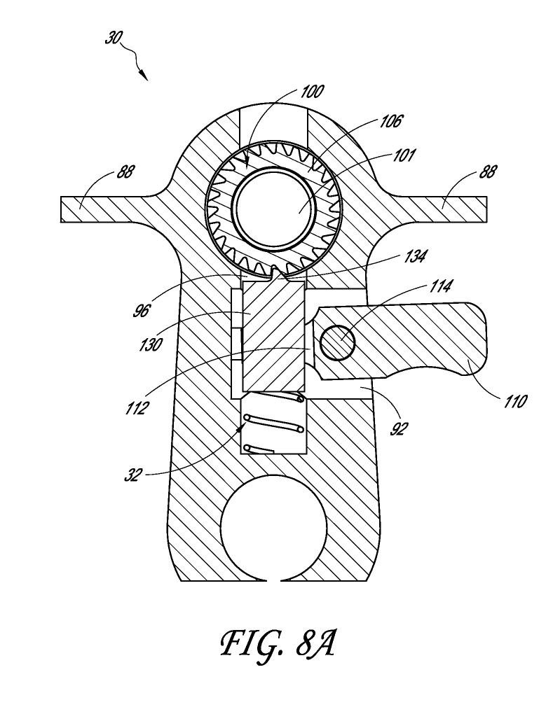

FIGS. 8A and 8B illustrate a mechanism that can prevent the retractor blade shaft 100 from unscrewing itself from the tulip of a pedicle screw. FIG. 8A is a top cross-sectional view of the lateral blade assembly 30, and FIG. 8B is a perspective view of the assembly with the retractor blade transparent for visibility. The blade assembly can include a locking pin 130 positioned at least partially within the pin bore 96. The locking pin can have a point 134 on one end that can fit within the locking grooves 106. A spring 32, also positioned within the pin bore 96, can bias the locking pin 130 into a locked position, in which the point 134 of the pin is in locking engagement with the locking grooves 106. In some embodiments, the locking grooves 106 can be angled relative to a diameter of the shaft bore 101 in order to create a ratcheting effect, such that when the pin is in a locked position the shaft 100 can rotate in a direction to screw into a tulip of a screw (typically clockwise), but is prevented from rotating in the opposite direction.

In some embodiments, the blade assembly 30 can have a lever 110 that can be used to manually release the locking pin 130 from the locked position, thus allowing the retractor blade shaft 100 to unscrew from its position within a pedicle screw. As illustrated in FIG. 8A, the lever 110 can be positioned within the lever bore 92. The lever bore can have an oblong shape, or at least shape that is larger than the lever 110 such that the lever can move within the lever bore. The locking pin 130 can have one or more gaps 132 (visible in FIG. 8B) bounded on either side by a surface, and an end 112 of the lever can be positioned within the gap. In the illustrated embodiment, the locking pin has a gap on an upper and a lower side of the locking pin, and the end of the lever is split into two prongs, one positioned within each gap. In some embodiments, the surfaces on either side of the gap can be curved.

The lever can be held in position by the lever pin 114, about which the lever can rotate. Consequently, pushing the lever toward the blade extensions 88 will cause the end of the lever 112 to rotate toward the spring 32. The end of the lever will push against a surface bounding a gap (or gaps) 32 of the locking pin 130 and push it toward the spring, releasing the locking pin 130 from the locked position and moving it into an unlocked position in which the retractor blade shaft 100 can freely rotate in either direction and can be unscrewed from the pedicle screw. When the lever is released the spring will tend to push the locking pin back into the locked position.

Returning to FIG. 7, a post 120 can be attached to the attachment section 90 of the retractor blade 80, typically on an upper surface of the attachment section. In some embodiments, the post can be inserted directly into the post attachment hole and held in place with a pin 126. The post can be used to attach the blade assembly 30 to the retractor, as discussed below. In some embodiments, the post can have a variety of shapes. In some embodiments, as illustrated, it can be generally rectangular. In some embodiments, the post can be generally cylindrical, oval, or of other shapes. In some embodiments, the post can have one or more detents to help align the post and blade assembly as desired.

Also visible in FIG. 7 is a cap 36, the top surface of which can be donut-shaped with a hole passing through its center, although the hole can be of any desired shape. The cap can be positioned in the central bore 86 around at least a portion of the retractor blade shaft 100, and can be attached (e.g., welded) onto the retractor blade shaft. In some embodiments, the hole in the cap can be aligned with the shaft bore 101. The outer perimeter of the cap can extend into the central bore 86 such that it touches or is adjacent to the ledge 87 (visible in FIG. 3), blocking the shaft from downward movement once the cap is attached to the shaft. So positioned, the top of the cap can be generally flush with an upper surface of the retractor blade 80. The cap can also have two curved cutouts 38 on opposite sides of each other. When the cap is attached to the retractor blade shaft, the cutouts 38 are preferably oriented at approximately 90 degrees from the notches 108 of the retractor blade shaft.

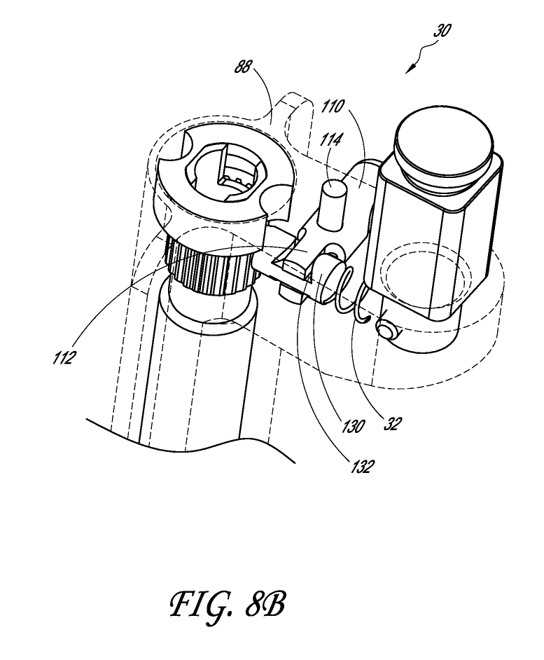

FIGS. 9A-11 illustrate a screw extender 140 and screwdriver 150. The screw extender can be used to attach the blade assembly to a pedicle screw, and the screwdriver can be used to insert the pedicle screw into a vertebra. As illustrated in FIG. 9A, a screw extender 140 can have at least two downward projections 148 that extend to the bottom of the screw extender opposite each other. The screw extender can also have two flexible sections 144, preferably positioned opposite each other as well, and preferably positioned such that each flexible section is located approximately 90 degrees about the screw extender from each downward projection. Each flexible section can be formed by a pair of cuts 145 on an outer surface 147 of the screw extender 140, the cuts extending along a portion of the length of the extender. In some embodiments, each pair of cuts can be parallel to each other.

FIG. 9B illustrates a side view of a screw extender 140, rotated 90 degrees from the view of FIG. 9A. FIG. 9C illustrates a cross-sectional view of FIG. 9B. As can be seen in FIG. 9B, the flexible sections 144 can have an equilibrium or locked position that is substantially flush with or angled slightly in from the outer surface 147 of the extender. However, the flexible sections can each have an outward extension 146 that extends beyond a plane of the respective outer surface 147 when the flexible sections are in the equilibrium or locked position. The flexible sections can be pushed inward into a central bore 142 (visible in FIG. 9C) until the flexible sections reach an unlocked position. In the unlocked position, the outward extensions 146 do not extend out past the planes of their respective outer surfaces 147.

When the flexible sections 144 are in the unlocked position, the screw extender can be inserted into the cap 36 of the blade assembly 30 (visible in FIG. 7). The downward projections 148 can fit within the curved cutouts 38, and the outward extensions 146 can fit within the central hole in the cap. Because the cap is oriented such that the cutouts are 90 degrees from the notches 108 of the shaft, and because the projections 148 are oriented 90 degrees from the outward extensions 146, the outward extensions will line up with the notches 108 of the shaft. When the flexible sections 144 are allowed to return to the equilibrium or locked position, the outward extensions will each extend into a notch 108 and below the top surface of the cap 36, thereby preventing removal of the screw extender 140 from its position in the cap. When the screw extender is in the cap, rotating the screw extender will rotate the retractor blade shaft 100, allowing it to be screwed into or out of the tulip of a pedicle screw.

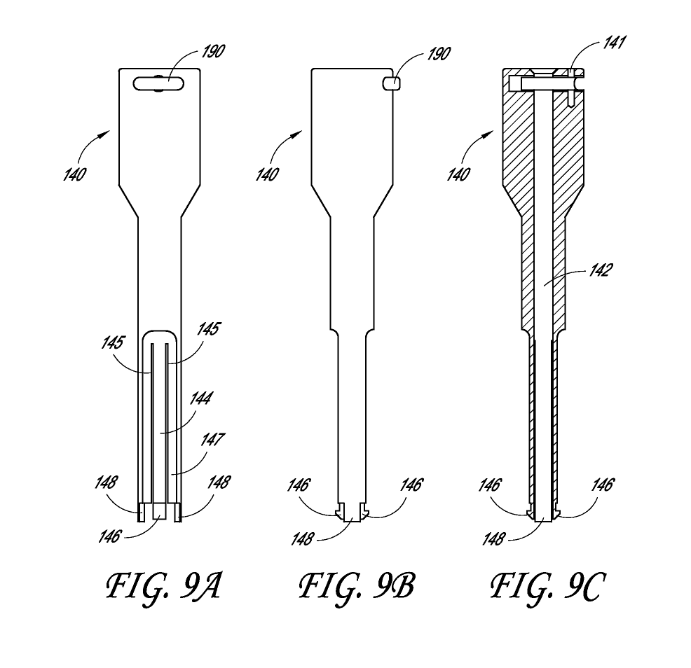

FIG. 10 is a side view of a screwdriver that can be used with the system described herein. The screwdriver is generally cylindrical, and is preferably sized such that it fits substantially flush within the central bore 142 of the screw extender 140. Thus, the screwdriver can occupy the available space within the central bore, preventing the flexible sections 144 from being pushed into the unlocked position. This can create a more solid connection between the screw extender and the blade shaft 100 while the screwdriver is being used.

The screw driver is preferably long enough to pass through the screw extender 140 and the bore 101 of the retractor blade shaft 100 in order to reach the shaft of a screw. The screwdriver has a distal tip 152 that can be configured to fit within a corresponding recess on the top of the screw shaft (e.g., a hex configuration), allowing the screwdriver to tighten the screw into a vertebra.

In some embodiments, it can be desirable to lock the screwdriver into a desired position within the screw extender 140 and blade shaft 100. This can be achieved by forming a plurality of annular recesses 154 on the outer surface of the screwdriver. The sections of the screwdriver with annular recesses can have a recessed diameter. The sections of the screwdriver between the annular recesses can have a standard diameter. The recesses can be used to lock the screwdriver against axial motion.

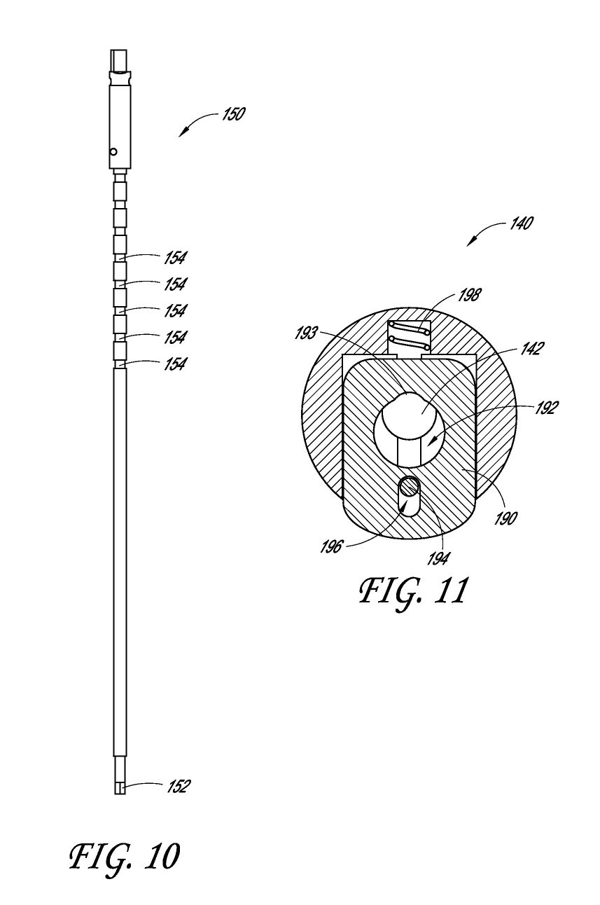

FIG. 11 is a top cross-sectional view of the screw extender 140 and illustrates a mechanism that can be used with the annular recesses to lock the screwdriver against axial motion. The screw extender can comprise a movable locking plate 190 with a generally circular cutout 192 and a positioning slot 196. The positioning slot can fit around a projection 194, which can help prevent the locking plate 190 from moving out of place. In some embodiments, the projection can be a pin inserted into a pin hole 141 (visible in FIG. 9C). In some embodiments, the cutout can have a section 193 with a smaller radius of curvature than the rest of the cutout.

The locking plate can block at least some of the central bore 142 of the screw extender. In an unlocked position, the locking plate is positioned such that at least sections of the screwdriver with the standard diameter can pass freely through. In a locked position, as illustrated in FIG. 11, the locking plate has blocked enough of the central bore 142 such that sections of the screwdriver with the standard diameter can no longer pass through.

In some embodiments, the screw extender can include a spring 198 or other biasing member, which can bias the locking plate into the locked position. Consequently, as the screwdriver is fed through the screw extender, when an annular recess passes through the cutout 192 of the locking plate 190 the locking plate will be pushed into the locked position. This will prevent further withdrawal or entry of the screwdriver, because the sections of standard diameter above and below the annular recess will be blocked by the locking plate.

The screwdriver can still rotate, however. In some embodiments, to improve the ability of the screwdriver to rotate when locked into the screw extender, the radius of curvature of the section 193 of the cutout can be approximately equal to half of the recessed diameter.

As visible in FIG. 11, and also FIG. 9B, the locking plate extends out of the interior of the screw extender, and can be manually pushed into the unlocked position. It can also be held in the unlocked position for easy insertion and removal of the screwdriver, or to position the screwdriver into a desired position before allowing the locking plate to lock the screw driver with a desired recess. The plurality of recesses can allow for locking the screwdriver in a desired position for use with blades and/or screw extenders of varying length.

In some embodiments, it can be desirable to have a screwdriver that has the ability for more refined axial locking. For example, in some embodiments, manufacturing tolerances in various components, such as the screw extender length, the depth of the recess in the screw, and/or the blade assembly length, can combine to make it such that the screwdriver does not properly engage the screw in any of its locked positions. This risk can be minimized by increasing the number of annular recesses in the screwdriver and decreasing their width.

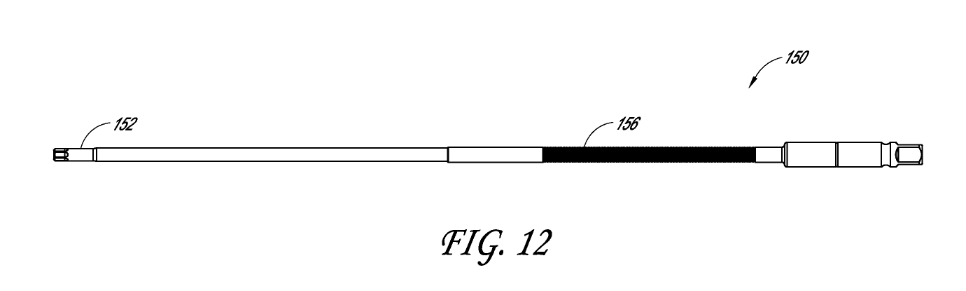

FIG. 12 illustrates one embodiment of a screwdriver that can minimize problems created from varying manufacturing tolerances. As illustrated, the screwdriver can have a section 156 with external ridges. The ridged section can help allow for a continuous span of lockable positions along the length of the ridged section. Thus, the screwdriver can be moved to adjust for any variance in height due to manufacturing tolerances and to ensure that the screwdriver is able to properly engage a screw. The screwdriver can also be adjusted for any variance in height requirements among different procedures. In some embodiments, the screwdriver can have a threaded section instead of or in addition to the section with external ridges.

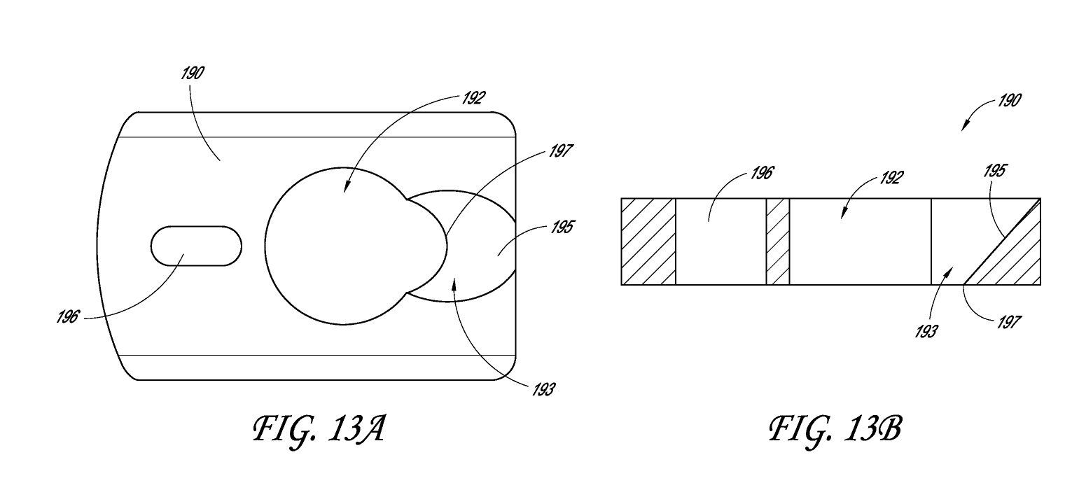

The screwdriver of FIG. 12 can be used with screw extenders as described above, including use of a locking plate to engage the screwdriver as described with respect to FIG. 11. In some embodiments, the locking plate can be modified to match the screwdriver. For example, FIGS. 13A and 13B illustrate one embodiment of a locking plate 190 configured for use with a ridged screwdriver. FIG. 13A is a top view and FIG. 13B is a side cross-sectional view. The locking plate can have a scalloped or trimmed portion to create an edge or tooth 197 in the section 193 with a smaller radius of curvature than the rest of the cutout 192. The tooth 197 can be sized and angled to engage the ridged section 156 and to prevent axial translation of the screwdriver when engaged. Thus, the screwdriver may not slide axially when locked, but it can still rotate, which can allow it to drive a screw forward or backward.

In some embodiments, the locking plate 190 and/or screwdriver 150 can receive a coating to increase its hardness and wear resistance. Coatings can include, for example, CrN, TiN, ZrN, a-C:H, etc. This can increase the life of the screwdriver and of the locking plate.

In some embodiments, a screw extender can have more than one locking plate 190 to engage the threaded section 156 of the screwdriver. This can help provide a more stable connection between the screw extender and screwdriver, while also increasing the durability of the system by sharing loads among multiple components. In some embodiments, having one or more locking plates engage the screwdriver on opposite sides can also help minimize loading on components by balancing loads on the screwdriver. This can further help the driver remain concentric with respect to the components of the screw extender.

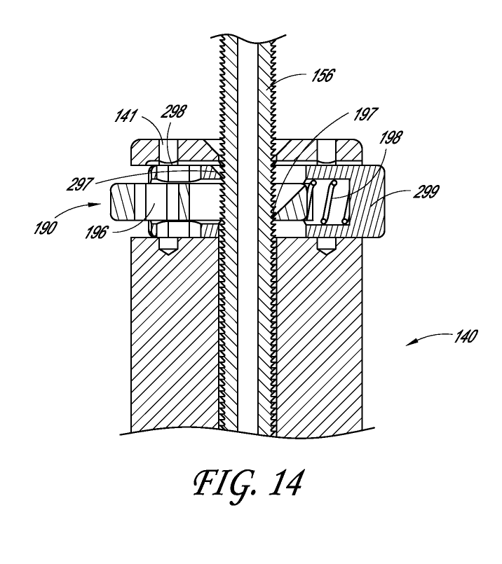

FIG. 14 illustrates a cross sectional view of one embodiment of a screw extender 140 that can lock the screwdriver by engaging it on multiple sides. The screw extender can include a locking plate 190 similar to the embodiment described with respect to FIGS. 13A and 13B. The locking plate can be positioned adjacent a spring 198 within a locking plate cover 299, which can also be configured to receive a screwdriver. Both the locking plate and locking plate cover can have slots 196, 298 respectively, which can receive a pin through a pin hole 141. This can allow the plate and cover to move within a range of positions relative to each other. In some embodiments, the locking plate cover can have an edge or tooth 297, similar to the edge or tooth 197 of the locking plate 190, and that generally faces the edge or tooth of the locking plate. The spring can be biased to move the locking plate and locking plate cover toward each other, which can push the teeth 197, 297 into engagement with the threaded section 156 of the screwdriver. A user can then push the locking plate and locking plate cover radially inward into the screw extender 140 to release the screwdriver and allow for its free axial motion.

Different arrangements are considered for engaging a screwdriver on multiple sides. For example, in some embodiments two separate springs can be used, each spring configured to engage a locking plate or a locking plate cover. In some embodiments, two locking plates can be positioned in the same plane on opposite sides of a screwdriver, and each locking plate can be spring biased into the screwdriver. Any other arrangement that biases a tooth into the screwdriver in more than one location can be used.

The various embodiments of screwdrivers and/or extenders described herein are not limited for use with a retractor system. They can be used in any surgical system that requires the use of a screwdriver and in which it may be beneficial to lock the screwdriver in a desired position. Such locking can, for instance, reduce the amount of toggle between the screw and screwdriver by ensuring a minimum engagement between the two. The various embodiments of screwdrivers and/or extenders described herein would have utility in any surgical system used to drive screws into bone, including but not limited to: pedicle screws such as for PLIF, TLIF, TPLIF and other surgeries; anterior cervical plates; posterior cervical systems; posterior lumbar systems; anterior lumbar systems such as standalone ALIF and ALIF plates; lateral plates; buttress plates; ISP plates; percutaneous screw systems; and other procedures.

### Anchor Extension Attachment Assemblies

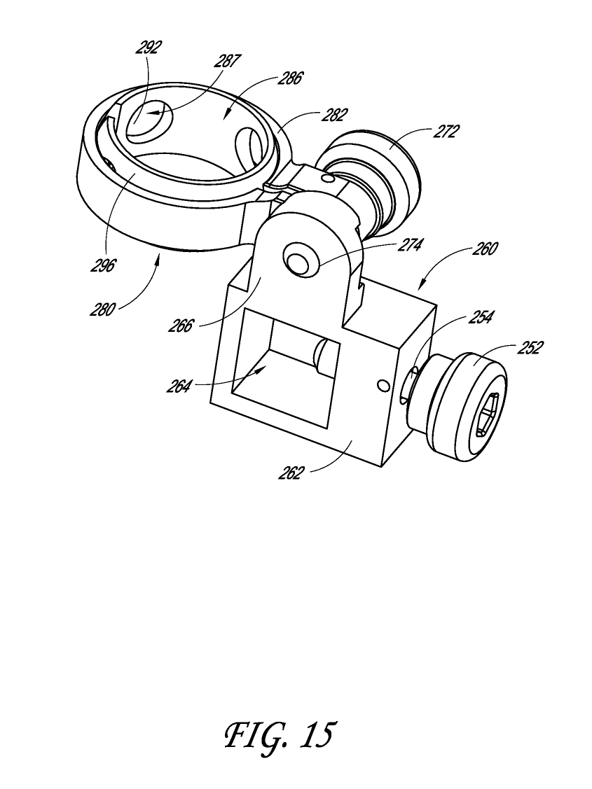

FIG. 15 illustrates one embodiment of an anchor extension attachment assembly 250, which can be used to attach different anchor extensions to a retractor system. The assembly can include an arm attachment portion 260 and a hoop assembly 280. The arm attachment portion can have at least one transverse cutout 264 that can be configured to fit around an arm of a retractor system 10. The arm attachment body 262 can also include an arm pin bore 254 that can be configured to receive a locking pin or screw 252. Thus, in some embodiments, the arm pin bore can have internal threading configured to match external threading of a locking screw 252. The arm attachment portion 260 can also include an extension 266 that can have a bore 274 configured to receive a hoop pin or screw 272. The hoop pin can be used to attach the hoop assembly 280 to the arm attachment portion 260. The hoop pin can also be used to tighten the hoop assembly around an anchor extension, such as an MIS tower.

In some embodiments, the hoop assembly 280 can be configured to attach to and tighten about an anchor extension that passes through the hoop assembly at varying angles. For example, in some embodiments the hoop assembly can have an outer ring or hoop 282 that at least partially surrounds an inner ring or hoop 286, as illustrated. In some embodiments, the inner ring or hoop 286 can have a curved outer surface 296 that can be configured to mate with a curved inner surface 292 of the outer ring or hoop 282. In some embodiments, the curved surfaces 296, 292 can have a generally equal radius of curvature, such that the inner hoop 286 can rotate relative to the outer hoop 282 prior to tightening the outer hoop. FIG. 1C illustrates one example of an inner hoop rotated relative to an outer hoop.

In some embodiments, the inner hoop can have one or more cutouts 287. The cutouts can minimize material requirements for the hoop assembly 280, they can improve the connection between the hoop and an anchor extension, they can help improve the flexibility of the inner hoop, and they can make the hoop assembly 280 easier to clean.

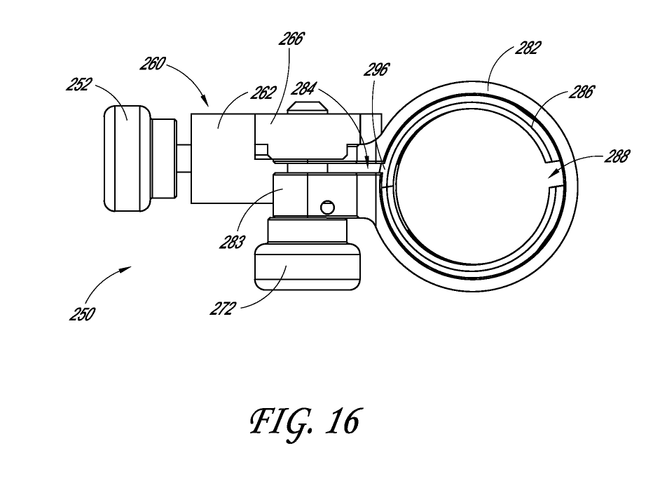

FIG. 16 illustrates a top view of an extension attachment assembly 250. In some embodiments, the outer hoop 282 can have a gap 284 and the inner hoop 286 can have a gap 288. Tightening the hoop pin or screw 272 can diminish the size of the gap 284 of the outer hoop, tightening the outer hoop around the inner hoop. This can tighten the inner hoop, which can help close the inner hoop gap 288. Closing the inner hoop gap can tighten the inner hoop about an anchor extension positioned through the inner hoop, such as an MIS tower.

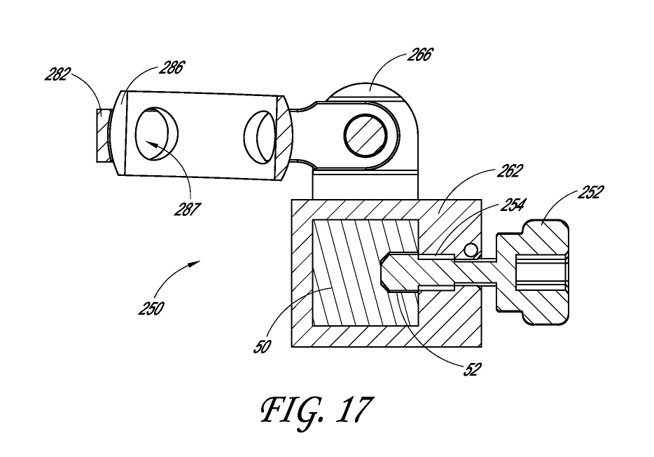

FIG. 17 is a cross-sectional view of an extension attachment assembly 250 as attached to an arm 50 of a retractor system 10. The arm attachment body 262 can be positioned around the arm 50, as further illustrated in FIGS. 1A and 1C. The arm locking pin or screw 252 can pass through the arm pin bore 254 and into a groove or slot 52 on an arm, which is also visible in FIG. 1C. Once the attachment assembly 250 has been moved to a desired position along the arm, tightening the pin or screw 252 into the groove 52 and against the arm 50 can lock the attachment assembly into position.

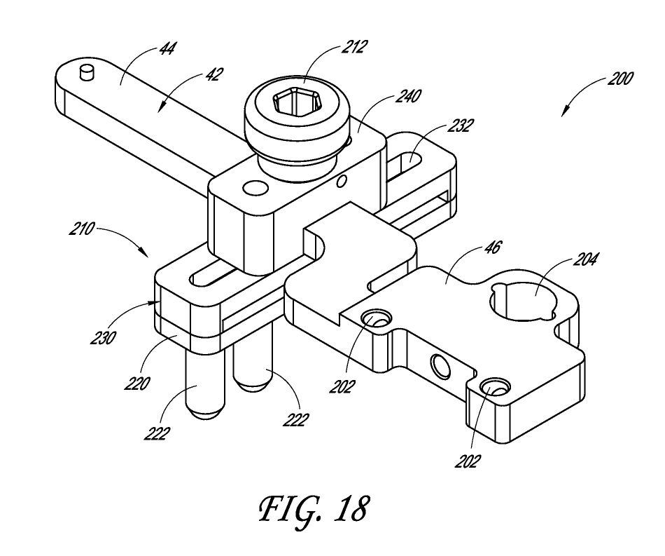

FIG. 18 illustrates one embodiment of a medial blade assembly 200. The medial blade assembly can include a medial blade attachment assembly 210 that includes an arm connecting section 220, which can be configured to attach the blade assembly to one or more arms of a retractor system. In some embodiments, a medial blade assembly can be configured to attach to any of the arms, and in some embodiments the blade assembly can be configured to attach to a single one of the arms. In some embodiments, a medial blade assembly 200 can be configured to connect to an arm 50 via one or more connecting pins 222 of the arm connection section 220, which can be positioned within one or more corresponding holes in an arm of the retractor system, such as the attachment holes 54 illustrated in FIG. 1A.

The medial blade attachment assembly 210 can also include a guiding section 230 that can be positioned next to the arm connecting section 220. In some embodiments, the arm connecting section and the guiding section can be integrally foamed. The guiding section 230 can be adjustably connected to a medial blade positioning section 240. For example, in some embodiments the guiding section 230 can have one or more grooves or notches 232 that can be positioned to receive one or more projections of the medial blade positioning section 240 or pins or screws attached to the medial blade positioning section. This can allow the positioning section to move relative to the guiding section along an axis of the grooves or notches.

A blade arm 42 can be attached to the medial blade positioning section 240. In some embodiments, the blade arm and positioning section can be integrally formed. In some embodiments, an adjustment section 44 of the blade arm can be slideably positioned within an opening in the positioning section 240. In such embodiments, the blade arm can be moved by sliding it along a longitudinal axis of the adjustment section through the opening in the positioning section 240. In some embodiments, the blade arm can be adjusted along two dimensions: moving the adjustment section 44 through the positioning section 240 along the longitudinal axis of the adjustment section, and moving the positioning section 240 and blade arm 42 along the longitudinal axis of the grooves or notches 232. Once the blade arm has been adjusted to a desired position, a pin or screw 212 can be tightened to lock the arm into the desired position.

The blade arm can also include a tool attachment section 46, to which tools can be attached before or after the blade arm has been moved into a desired position. The tool attachment section can have a variety of holes having a variety of configurations allowing the holes to receive a variety of tools. For example, the tool attachment section can have one or more light attachment holes 202 that can receive a lighting component 2, such as described with respect to FIG. 1A. The tool attachment section 46 can additionally or alternatively have one or more blade attachment holes 204 that can each be configured to receive a medial retractor blade.

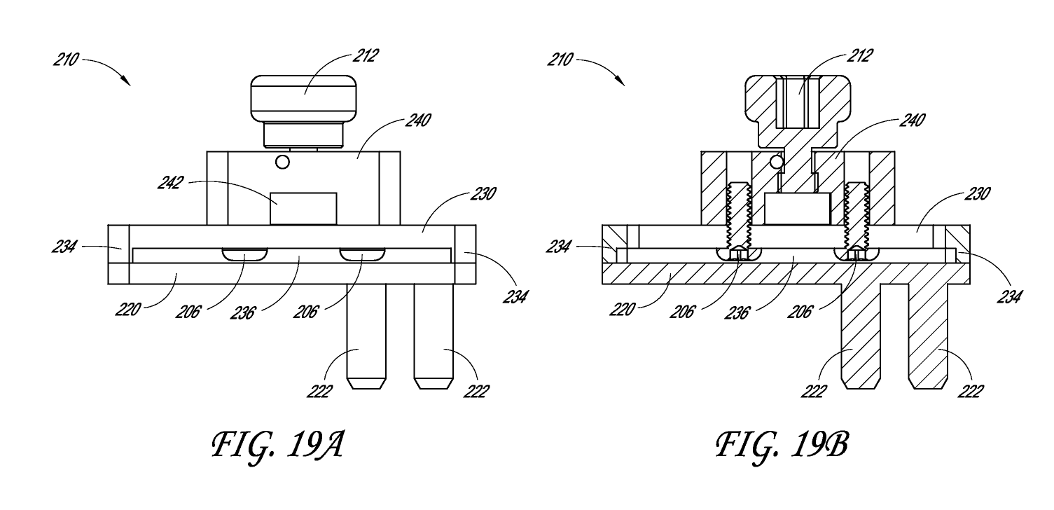

FIG. 19A illustrates a side view of a medial blade attachment assembly and FIG. 19B illustrates a cross-sectional view in the same plane. As illustrated, in some embodiments the medial blade positioning section 240 can have a cutout 242 sized and configured to receive a blade arm 42. In some embodiments, the guiding section 230 can have extensions or legs 234 on either side which can create a space 236 between the guiding section and the arm connecting section 220. The space can be sized to fit the heads of screws 206, which can be used to attach the guiding section 230 to the medial blade positioning section 240. The shafts of the screws can pass through the groove or notch 232 and can serve as rails or guideposts for translation of the medial blade positioning section 240 along the longitudinal axis of the groove or notch.

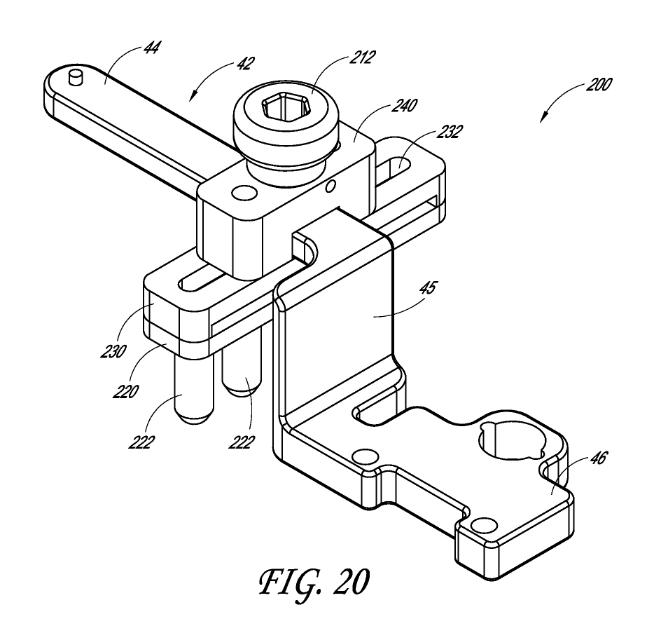

In some embodiments, as illustrated in FIG. 20, blade arm 42 can have different sizes or configurations depending upon an expected desired positioning of the tool attachment section 46. For example, in some embodiments the blade arm can have one or more bends 45 which can affect the vertical positioning of the tool attachment section. In some embodiments, a bend can be used to lower the tool attachment section relative to the adjustment section 44. In some embodiments, a bend can be used to elevate the tool attachment section relative to the adjustment section.

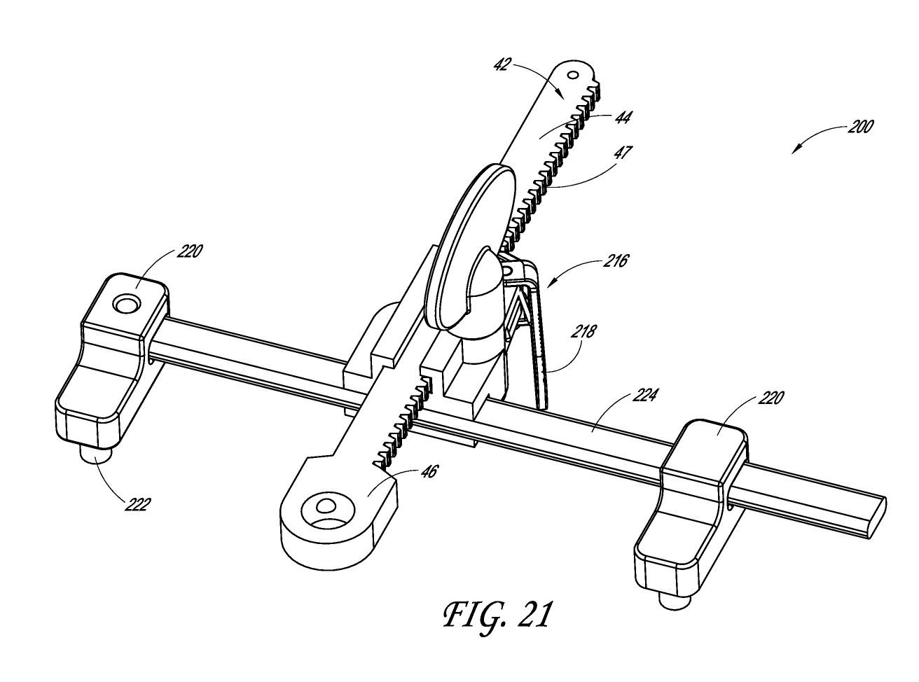

FIG. 21 illustrates an embodiment of a medial blade assembly 200 that has two arm connecting sections 220, each adapted to attach to a different arm. A medial blade cross bar 224 can join the connecting sections, and the cross bar can be slidably connected to at least one of the connection sections 220. This can allow for the arms to move relative to each other with the medial blade assembly attached to the arms. A ratchet assembly 216 can attach to the cross bar 224. In some embodiments, the ratchet assembly can be movable along the length of the cross bar. In some embodiments, the ratchet assembly can be fixed relative to the cross bar.

A blade arm 42 can attach to the ratchet assembly 216. The blade arm can have a tool attachment section 46 at one end and an elongate adjustment section 44 that extends from the attachment section. The adjustment section can have teeth 47 that can engage with a lever or latch 218 of the ratchet assembly 216 to lock the position of the adjustment section relative to the ratchet assembly. In some embodiments, the lever or latch can lock the position of the adjustment section in only one direction. In some embodiments, the lever or latch can allow the adjustment section to move relative to the ratchet assembly such that the tool attachment section 46 moves toward the ratchet assembly 216, but lock the adjustment section from moving such that the tool attachment section moves away from the ratchet assembly.

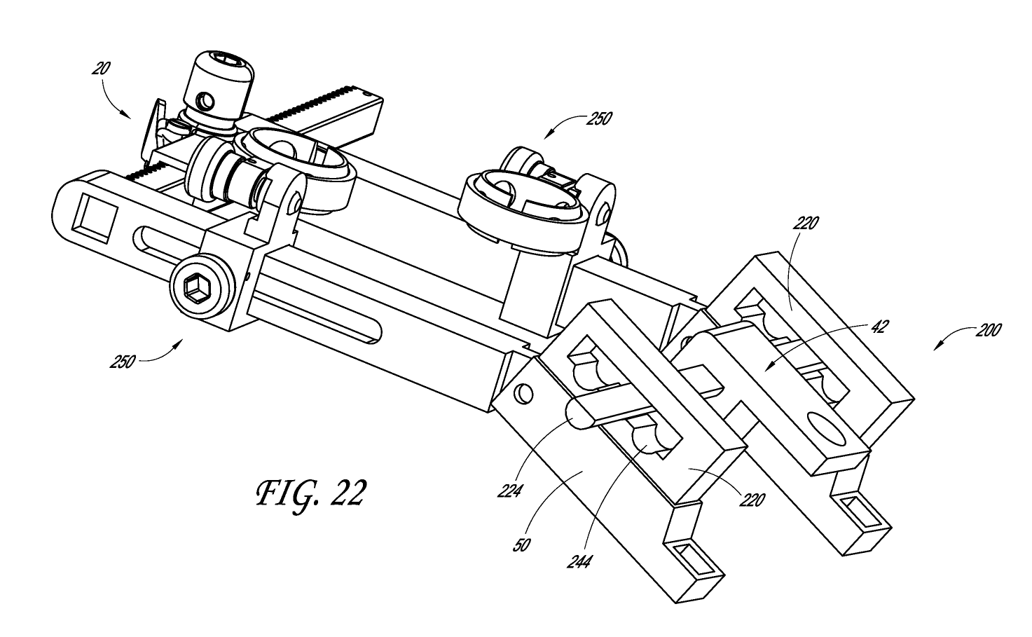

In some embodiments, in addition to allowing adjustment of a blade arm 42 position in two dimensions, a medial blade assembly can be configured to allow angular rotation of the blade arm 42. FIG. 22 illustrates an embodiment of a medial blade assembly 200 that has a cross bar 224 with a rounded lower surface. The cross bar can fit within curved cutouts 244 on an arm connecting section 220. The curved cutouts can be configured to match the curvature of the cross bar. The cross bar can rotate within the curved cutouts, adjusting the angle of the blade arm 42 about an axis of the cross bar 224. In some embodiments, as illustrated, the arm connecting section 220 can have a plurality of curved cutouts 244, and the position of the blade arm 42 can be adjusted by locating the cross bar in different curved cutouts.

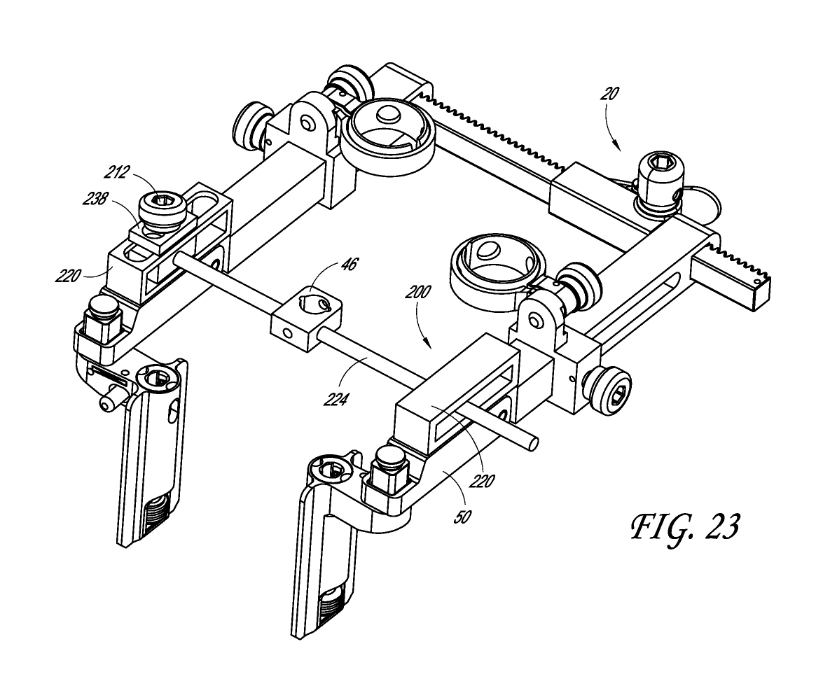

FIG. 23 illustrates an embodiment of a medial blade assembly 200 with a cylindrical cross bar 224 that has each end passing through an arm connection section 220. Rotating the cross bar can adjust the angle of a tool attachment section 46 connected to the cross bar. The cross bar can also be configured to slide within a side slot of the arm connection section 220, drawing the tool attachment section 46 closer to or farther away from each arm. In some embodiments, at least one end of the cross bar 224 can be attached to a medial blade positioning section 240 positioned within the arm connecting section 220. The blade positioning section can be configured to move along a longitudinal axis of a retractor arm 50. In some embodiments, one or more screws or pins 212 can pass into the arm connecting section and blade positioning section. Tightening the one or more screws or pins can lock the cross bar 224 into its current position. In some embodiments, a brace 238 can be positioned between the arm connecting section 220 and a head of one or more of the pins or screws 212 to help create a tighter connection.

### Minimally Invasive Tower Access Device

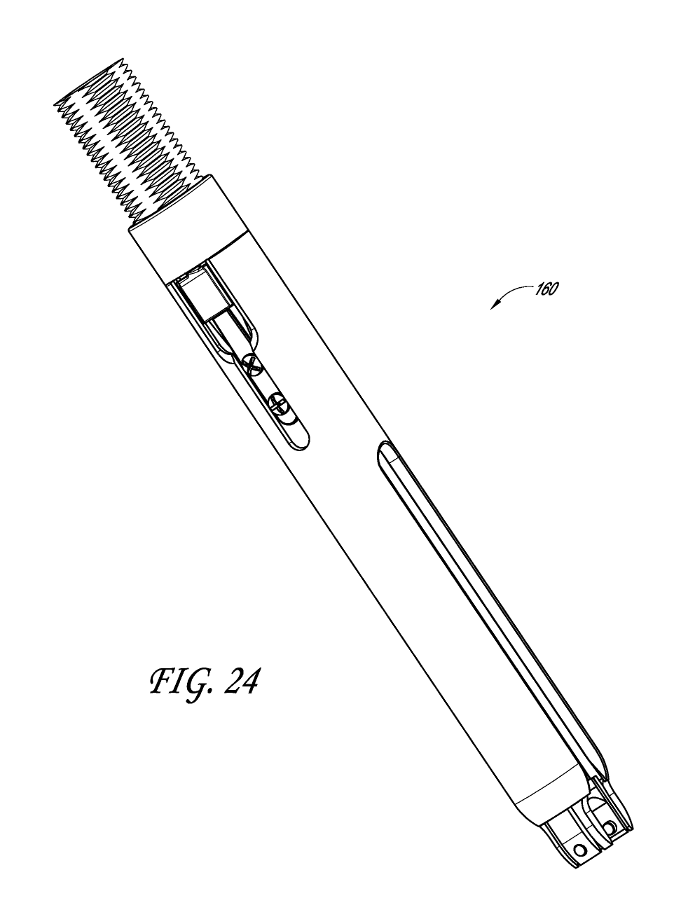

FIG. 24 illustrates one embodiment of a minimally invasive tower access device 160 that can be used as described with respect to various embodiments described herein. For example, the tower 160 can be used to deliver a spinal screw to a location proximate to a bone member where the spinal screw can be inserted. A tower can also serve as a portal or opening extending from the bone member to outside of the patient, through which instruments and implants (such as rods) can be delivered. In some embodiments, towers can be used to attach pedicle screws to the pedicles of vertebrae adjacent an intervertebral space to be operated on. Towers are described in more detail in U.S. Provisional Patent Application No. 61/653,853, filed on May 31, 2012, and U.S. Patent Application Publication No. 2012/0022594 A1, published Jan. 26, 2012, both of which are hereby incorporated by reference in their entireties and are to be considered a part of this specification.

### Retractor

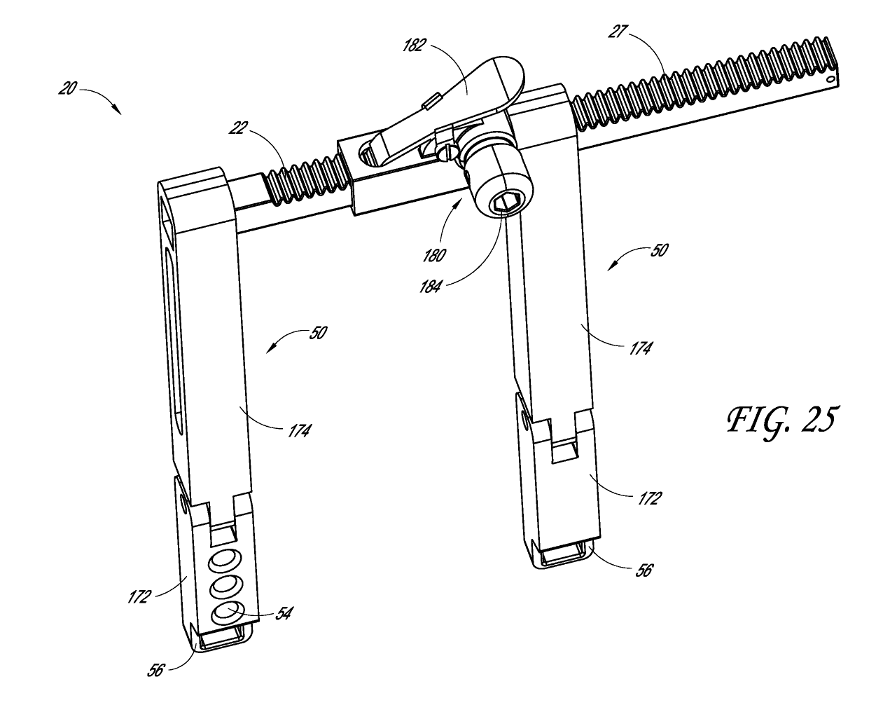

FIG. 25 illustrates one embodiment of a retractor body 20 that can be used with a MAS retractor system. As described above, the arms 50 can be attached to a variety of anchor extensions, such as lateral blade assemblies and MIS towers. Once the arms 50 are attached to the desired anchor extensions, the arms and anchor extensions can be moved apart, which will tend to distract the disc space about which the extensions are attached. The arms can move according to various embodiments. As illustrated, the retractor body 20 can include a cross bar 22 with ridges or teeth 27. Both arms can be attached to the cross bar. In some embodiments, one arm can be attached with a moveable latch mechanism 180, which can have a latch 182 that can be spring-biased to lockingly engage the notches in the cross bar. Pushing on the latch can release it, allowing the arms to be moved closer together or farther apart. In some embodiments, the arms can be manually moved closer together or farther apart. In some embodiments, an adjustment screw 184 can be turned to help move the arm attached to the latch mechanism 180 along the cross bar 22.

The arms 50 can include a collar 56 that is sized to fit over the post 120 of a blade assembly 30 (e.g., the post visible in FIG. 7). Preferably, the collar is oriented such that when a blade assembly is positioned within the collar, the blade plane (described above) is either generally parallel to or generally perpendicular to the cranial-caudal line of the patient or the cross bar 22 of the retractor body 20.

In some embodiments, the arms 50 can comprise a plurality of sections joined by articulating joints, and the most lateral sections can be configured to attach to the lateral blade assembly. In some embodiments, the arms 50 have a first section 172 and a second section 174, with the first section configured to connect to a lateral blade assembly. As illustrated, the first section 172 can articulate relative to the second section. In some embodiments, articulation can be locked, such as through the use of a screw, locking pin, or other device to hold the sections of the arms at a desired angle. The articulating arms allow the collar 56 to fit over the post 120 of a blade assembly 30 when the blade assembly has been angled away from the spine (such as when the surgeon desires to perform a TLIF) while still allowing the cross bar 22 to remain flat against the patient. This can minimize interference with or undesired forces on any anchor extensions attached to the retractor on the contralateral side and/or to the second section 174 of the arms 50.

In some embodiments, as described above, the retractor body can be configured to connect to a medial blade assembly 200 (such as that visible in FIG. 1) that can position a medial blade 40 between lateral blade assemblies 30. As discussed above, the medial blade can be configured to move medially, riding up the spinous process, to retract tissue and open a visual and operational space for the procedure. The medial blade assembly preferably attaches to the first section 172 of the arms 50, such that it can be positioned at the same angle as the lateral blade assemblies 30. Having all of the blades aligned at the same angle can help minimize damage to the multifidus muscle when the disc space is accessed. In some embodiments, particularly when a TLIF is the desired procedure, the blades can be positioned at an angle between 25 degrees (or about 25 degrees) and 30 degrees (or about 30 degrees). Other angles and approaches are considered. In some embodiments, as described above, the medial blade assembly 200 can be configured to allow a medial blade 40 to rotate independently of the angle of the arms 50.

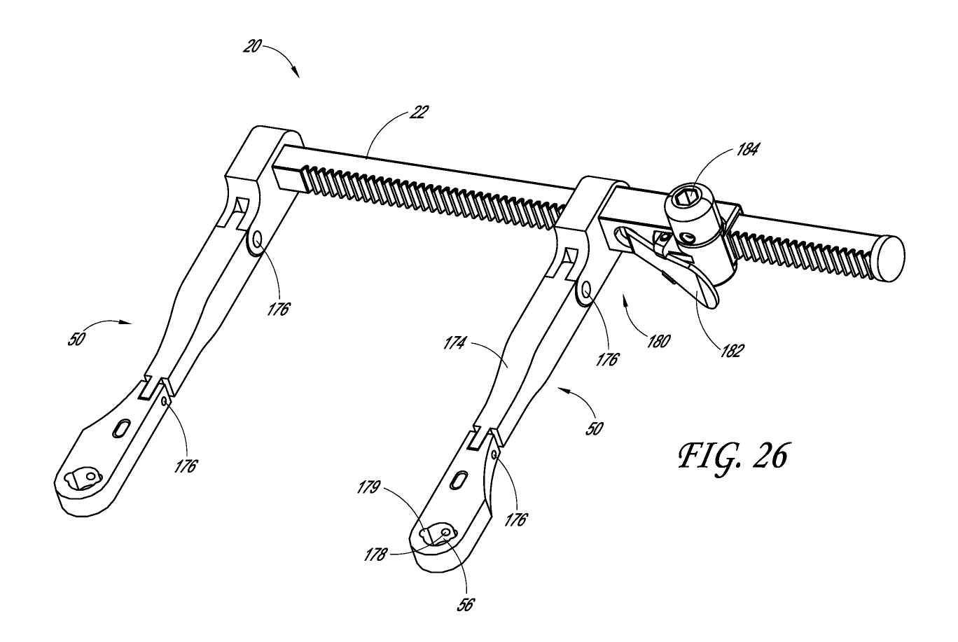

FIG. 26 illustrates an embodiment of a retractor body in which the second section 174 of the retractor arms 50 can rotate relative to the cross bar 22 in addition to being able to rotate relative to the first section 172. FIG. 26 also illustrates screws or pins 176 that can be used to tighten and/or lock one or more of the sections of the arms 50 relative to adjacent sections.

In some embodiments, the collar 56 can be configured to receive a generally round or other shaped blade assembly post. The collar can include a plurality of recesses 178 or slots 179 spaced about the interior circumference of the collar. Preferably, there are four recesses or slots spaced 90 degrees apart about the circumference, and at least one pair of opposing slots are on a line that is parallel to the cranial-caudal line of the patient, or the cross bar 22 of the retractor body 20. In some embodiments, the post and the blade assembly can rotate within the collar, and a detent on a blade assembly post can snap into position within the recesses 178 or slots 179. The positioning of the detent and recesses or slots 179 can be such that when the detent has snapped into position the blade plane is either generally parallel to or generally perpendicular to the cranial-caudal line of the patient, or the cross bar 22 of the retractor body 20.

### Methods for Accessing Disc Space

Various embodiments of methods of using a retractor system to insert a spinal implant within a vertebral disc space are described. To begin, a surgeon marks the positions on a patient's back that lie above both pedicles of the vertebrae on either side of the desired disc space. Using techniques known in the art, an incision is created on each marked spot. Either now, or later in the procedure, the surgeon can join the incisions on the same side of the spine. The surgeon can then use his or her finger to separate the muscle along the incision, preferably dissecting it along a single plane to make the healing process quicker and easier.

A drill guide can be placed through the incision and onto the entrance to a pedicle. A drill can be advanced through the drill guide to drill a hole in the pedicle. A guide wire can then be inserted through the cannula of the drill guide and into the pedicle, and the drill guide can be removed. In some embodiments, the guide wire can be inserted through trocars, needles, or other hollow instruments instead of the drill guide.

The surgeon can select the desired bone screw and appropriate length lateral blade assemblies. The surgeon can attach a blade extender to the blade assembly and then attach the blade assembly to a screw, as described above. Inserting the screwdriver through the screw extender and into the blade assembly can lock the screw extender into place, as discussed above, and the combination of the attached screw, blade, extender, and screwdriver can be inserted along the guide wire and into position on a pedicle on the operational side. In some embodiments, the screw can be inserted first and then the blade and screw extender can be inserted to attach the blade to the screw. In some embodiments, one or more dilators can be inserted prior to inserting the blade assemblies in order to expand a space for insertion of the blade assemblies. In some embodiments, the screw can be attached to other anchor extensions such as MIS tower assemblies.

Generally, when the blade is first inserted into the patient it is oriented such that the blade plane (defined above) is parallel to the opening joining the incisions (i.e. generally parallel to the spine). Because the retractor blade shaft can rotate independent of the retractor blade, as discussed above, if the blade assembly is attached to the screw after the assembly has been positioned within the patient, the retractor blade will be able to maintain its orientation relative to the patient.

Once the blade, screw, extender, and screwdriver are in place, the screwdriver can be used to drive the screw into the pedicle. Only the screw shaft and the screwdriver will rotate, and the remaining components will maintain their orientation relative to the patient. Once the screw has been fully inserted into the bone, the screwdriver can be removed and then the extender can be removed. The blade assemblies can be rotated 90 degrees (either before or after removing the blade extender), such that the blade plane is generally perpendicular to the spine. This will retract tissue and create a visual and operational space in which the surgeon can operate. The guide wire can be removed at any point after the screw has begun to enter the pedicle.

This procedure is then repeated for the opposite pedicle on the operational side. Additionally, if desired by the operating surgeon, pedicle screws can be inserted with towers into the opposite pedicles on the contralateral side either before or after inserting the lateral blade assemblies on the operational side. As discussed above, additional arrangements of anchor extensions can be used, such as using MIS towers instead of lateral blade assemblies.

Once all desired pedicle screws have been placed (e.g., two with blade assemblies on the operational side and two with towers on the contralateral side), the retractor body can be positioned over the patient's back and attached to the towers and lateral blades. If the surgeon desires to adjust the angle of the lateral blades, this can be done without affecting the positioning of the retractor body due to the articulated arms, as discussed above. In some embodiments, the surgeon can rotate the lateral blade assemblies by approximately 90 degrees to help establish an operational corridor. This can be done either before or after attaching the blades to the retractor body, or before or after adjusting the angle of the blades. In some embodiments, once an operational corridor has been established a surgeon may desire to improve access to an intervertebral space, such as by performing a facetectomy. The retractor body can distract the disc space by moving the arms of the retractor apart.

If desired, the surgeon can position a medial blade between the two lateral blades and attach the medial blade to the retractor. The medial blade can then be moved medially, retracting more tissue and broadening the visual and operational space. The standard TLIF, PLIF, TPLIF, or other procedures can then be performed. Following the procedure, the blades can be unscrewed from the pedicle screws in the same manner in which they were attached. If desired, rods can be inserted to join the pedicle screws, and the rods can be locked into place with set screws, as discussed above. Rods can also be inserted to join pedicle screws on the contralateral side, and the towers can be removed from the pedicle screws on that side.

If the surgeon desires to perform the procedure on multiple adjacent levels, then additional screws can be inserted in the next level using the same methods described above. One advantage of this system is that the lateral blade assemblies can be symmetrical and can be used on either the cranial or caudal side of the operation (also referred to as the left or right side, from the surgeon's point of view). Thus, the blade adjacent the newly inserted pedicle screw on the operational side does not need to be removed and replaced, but can instead be rotated 180 degrees. The procedure can then proceed as described above.

Although the foregoing description of the preferred embodiments has shown, described and pointed out the fundamental novel features of the invention, it will be understood that various omissions, substitutions, and changes in the form of the detail of the apparatus as illustrated as well as the uses thereof, may be made by those skilled in the art, without departing from the spirit of the invention.

Reference throughout this specification to “one embodiment” or “an embodiment” means that a particular feature, structure or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures or characteristics of any embodiment described above may be combined in any suitable manner, as would be apparent to one of ordinary skill in the art from this disclosure, in one or more embodiments.

Similarly, it should be appreciated that in the above description of embodiments, various features of the inventions are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Rather, as the following claims reflect, inventive aspects lie in a combination of fewer than all features of any single foregoing disclosed embodiment. Thus, the claims following the Detailed Description are hereby expressly incorporated into this Detailed Description, with each claim standing on its own as a separate embodiment.

What is claimed is:

1. A minimally invasive surgical system for treating the spine, comprising:
at least one blade assembly comprising:
a blade having a central section with a central bore and wings extending outward from the central section on opposite sides thereof, said wings extending to a location below a bottom of the central section, forming a gap bounded by the wings on two sides and by the bottom of the central section on a third side;
a post extending from an upper surface of the blade; and
a shaft positioned at least partially within the central bore of the blade, the shaft comprising an externally threaded lower section and a central shaft bore that runs through the length of the shaft, wherein the shaft is rotatable within the central bore of the blade.

2. The minimally invasive surgical system of claim 1, wherein the blade shaft further comprises a plurality of vertical grooves extending around at least a portion of the shaft.

3. The minimally invasive surgical system of claim 2, wherein the blade assembly further comprises a locking pin positioned within the blade, the locking pin moveable between a locked position in which a point on one end of the locking pin engages at least one of the vertical grooves of the blade shaft, substantially preventing rotation of the shaft, and an unlocked position in which the point is disengaged from the vertical grooves.

4. The minimally invasive surgical system of claim 3, wherein the locking pin is biased toward the locked position.

5. The minimally invasive surgical system of claim 3, wherein the vertical grooves are angled relative to a diameter of the shaft bore such that the locking pin in the locked position only prevents rotation of the shaft in one direction.

6. The minimally invasive surgical system of claim 1, wherein the externally threaded lower section extends at least partially into the gap.

7. The minimally invasive surgical system of claim 6, further comprising a pedicle screw comprising a screw shank and a housing attached to an upper end of the screw shank, the housing having internal threading configured to receive the external threads of the lower section of the blade shaft.

8. The minimally invasive surgical system of claim 7, further comprising a retractor comprising a cross bar with a plurality of arms extending from the cross bar, wherein at least one of the arms can move relative to another one of the arms to change a distance between the arms.

9. The minimally invasive surgical system of claim 8, wherein each arm comprises at its end farthest from the cross bar a collar configured to attach to the post of the blade assembly.

10. The minimally invasive surgical system of claim 8, wherein at least two of the arms are configured to attach to a minimally invasive tower access device.

11. The minimally invasive surgical system of claim 8, further comprising a medial blade configured to attach to the retractor.

12. A minimally invasive surgical system for treating the spine, comprising:
a plurality of pedicle screws for insertion into the pedicles of adjacent vertebrae, each of the pedicle screws having an upper portion that is internally threaded;
a plurality of retractor blades, each of the blades having an externally threaded lower section configured to engage the internally threaded upper portion of a corresponding pedicle screw; and
a plurality of retractor arms configured to move the retractor blades closer together or farther apart.

13. The minimally invasive surgical system of claim 12, further comprising a plurality of minimally invasive tower access devices configured to engage at least some of the plurality of pedicle screws.

14. The minimally invasive surgical system of claim 13, wherein the plurality of retractor arms includes at least some retractor arms configured to engage the plurality of minimally invasive tower access devices to move the minimally invasive tower access devices closer together or farther apart.

15. The minimally invasive surgical system of claim 12, further comprising an implant configured to be delivered between the plurality of retractor blades.

16. A minimally invasive surgical method for treating the spine, the method comprising:
delivering a first pedicle screw into the pedicle of a first vertebra and a second pedicle screw into the pedicle of a second vertebra, wherein the first and second pedicle screws have threaded upper portions;
distracting a disc space between the first and second vertebra by moving retractor blades engaged with the threaded upper portions of the first and second pedicle screws; and
delivering an implant between the retractor blades into the disc space.

17. The method of claim 16, further comprising delivering a third pedicle screw into the pedicle of the first vertebra on a contralateral side relative to the first pedicle screw, and delivering a fourth pedicle screw into the pedicle of the second vertebra on a contralateral side relative to the second pedicle screw.

18. The method of claim 17, wherein the disc space is distracted at least in part by moving screw extensions engaged with the third and fourth pedicle screws.
